(19) United States
(12) Patent Application Publication
(10) Pub. No.: US 2024/0383839 A1
(43) Pub. Date: Nov. 21, 2024

(54) BIO-BASED METHYL DIHYDROJASMONATE, BIO-BASED CYCLOPENTANONE, PROCESS FOR THEIR PREPARATION AND USE THEREOF

(71) Applicant: SPECIALTY OPERATIONS FRANCE, Lyon (FR)

(72) Inventors: Zhen Yan, Taiyuan (CN); Chenglu David, Saint-Fons (FR)

(73) Assignee: SPECIALTY OPERATIONS FRANCE, Lyon (FR)

(21) Appl. No.: 18/833,783

(22) PCT Filed: Jan. 31, 2023
(86) PCT No.: PCT/EP2023/052268

§ 371 Date: Jul. 26, 2024

(30) Foreign Application Priority Data
Jan. 31, 2022 (WO) PCT/CN2022/075273

(51) Int. Cl. C07C 69/716; C07C 49/395

(52) U.S. Cl. CPC C07C 69/716; C07C 49/395; C07C 2601/08

(57) ABSTRACT

The present invention relates to a new bio-based methyl dihydrojasmonate and cyclopentanone. Methyl dihydrojasmonate having a bio-based carbon content greater than or equal to 30% and/or a number of carbon atoms from bio-based origin greater than or equal to 2, and cyclopentanone having a bio-based carbon content higher or equal to 50% or a number of carbon atoms from bio-based origin greater than or equal to 2 are described. The invention further relates to a process for their preparations and the use of such compounds.

This application claims priority filed on 31 Jan. 2022 with PCT/CN2022/075273, the whole content of this application being incorporated herein by reference for all purposes.

### TECHNICAL FIELD

The present invention relates to a bio-based methyl dihydrojasmonate, a bio-based cyclopentanone, a process for their preparation and use thereof.

### BACKGROUND

Methyl dihydrojasmonate (CAS 24851-98-7) is an aroma compound whose odor is floral and citrus in racemic mixtures. The compound is used in perfumery and in the food industry. Methyl dihydrojasmonate is utilized as synthetic equivalent to methyl jasmonate, a component of naturally occurring jasmine. Industrially, methyl dihydrojasmonate may be produced from adipic acid as precursor for the preparation of cyclopentanone. Cyclopentanone can then be functionalized by aldol condensation with pentanal followed by a Michael addition of dimethyl malonate.

Synthetic flavourings tend to be less well liked by consumers than flavourings of natural origin. There is thus a growing interest in other sources of methyl dihydrojasmonate and in particular routes using natural raw material that can be labelled either natural or bio-sourced according to existing legislations.

### BRIEF DESCRIPTION OF THE INVENTION

In a first aspect, the present invention relates to methyl dihydrojasmonate having a bio-based carbon content higher or equal to 30%, or wherein the number of carbon atoms from bio-based origin is higher or equal to 5.

In another aspect, the present invention relates to a process for the preparation of methyl dihydrojasmonate from bio-based furfuryl alcohol whose bio-based carbon content is higher or equal to 50%.

The present invention further relates to a bio-based cyclopentanone having a bio-based carbon content higher or equal to 50%.

In another aspect, the present invention relates to a bio-based cyclopentanone having at least 2 bio-based carbon atoms.

In another aspect, the present invention relates to a process for the preparation of cyclopentanone from bio-based furfuryl alcohol whose bio-based carbon content is higher or equal to 50%.

The present invention further refers to the use of a cyclopentanone or a methyl dihydrojasmonate according to the present invention as flavor or fragrance, in particular its use in perfumery, cosmetics, pharmacy.

Finally the invention also relates to a composition comprising a methyl dihydrojasmonate and/or cyclopentanone according to the invention, preferably selected from the group consisting of food products, beverages, cosmetic formulations, pharmaceutical formulations, fragrances.

### DETAILED DESCRIPTION OF THE INVENTION

The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The term “and/or” includes the meanings “and”, “or” and also all the other possible combinations of the elements connected to this term.

Throughout the description, including the claims, the term “comprising one” should be understood as being synonymous with the term “comprising at least one”, unless otherwise specified, and “between” should be understood as being inclusive of the limits. It is specified that, in the continuation of the description, unless otherwise indicated, the values at the limits are included in the ranges of values which are given.

The expression “comprise” should be understood as including equally “consist of” or “consist substantively of”.

It should be noted that in specifying any numerical range, such as a range of concentration, conversion rate or selectivity, any particular upper limit can be associated with any particular lower limit.

If not specified otherwise, a percentage content is on weight basis.

In the present invention, the expressions “bio-based material”, “bio-sourced material” or “natural material” designate a product that is composed, in whole or in significant part, of biological products or renewable agricultural materials (including plant, animal, and marine materials) or forestry materials.

In the present invention, the expression “bio-based carbon” refers to carbon of renewable origin like agricultural, plant, animal, fungi, microorganisms, marine, or forestry materials living in a natural environment in equilibrium with the atmosphere. The bio-based carbon content is typically evaluated by the means of the carbon-14 dating (also referred to as carbon dating or radiocarbon dating). Furthermore, in the present invention, the “bio-based carbon content” refers to the molar ratio of bio-based carbon to the total carbon of the compound or the product. The bio-based carbon content can preferably be measured by a method consisting in measuring decay process of 14C (carbon-14), in disintegrations per minute per gram carbon (or dpm/gC), through liquid scintillation counting, preferably according to the Standard Test Method ASTM D6866-16. Said American standard test ASTM D6866 is said to be equivalent to the ISO standard 16620-2. According to said standard ASTM D6866, the testing method may preferably utilize AMS (Accelerator Mass Spectrometry) along with IRMS (Isotope Ratio Mass Spectrometry) techniques to quantify the bio-based content of a given product.

In the present invention, the expression “813C” refers to the mean isotopic deviation of carbon-13. The mean isotopic deviation of ¹³C is measured by Isotope Ratio Mass Spectrometry using PDB as international standard. The isotopic fingerprint of a compound provides information on the origin of said compound in particular natural or fossil.

### Methyl Dihydrojasmonate

The invention relates to methyl dihydrojasmonate having a bio-based carbon content higher or equal to 30%, preferably higher or equal to 35%, more preferably higher or equal to 38%. Generally methyl dihydrojasmonate according to the present invention has a bio-based carbon content lower or equal to 105%, preferably equal to lower to 100%, more preferably lower than 100%.

According to another aspect, the number of carbon atoms from bio-based origin of methyl dihydrojasmonate according to the present invention is higher or equal to 2, preferably higher or equal to 5, more preferably higher or equal to 7. In a specific embodiment, all carbon atoms of methyl dihydrojasmonate according to the present invention are from bio-based origin.

The present invention further relates to a composition comprising, or consisting essentially of methyl dihydrojasmonate, whose bio-based carbon content is higher or equal to 30%, preferably higher or equal to 35%, more preferably higher or equal to 38.

Said methyl dihydrojasmonate may represent the major compound of the composition according to the present invention. Accordingly said methyl dihydrojasmonate may represent more than 50%, preferably more than 70%, more preferably more than 80% regarding the total weight of the composition. In a more preferred aspect of the present invention the said methyl dihydrojasmonate may represent more than 90%, preferably more than 95%, more preferably more than 96%, more preferably more than 99%, most preferably more than 99.5% regarding the total weight of the composition. The impurity may represent from 1 ppm to 5000 ppm, preferably from 1 ppm to 500 ppm, more preferably from 1 ppm to 50 ppm, most preferably from 1 ppm to 20 ppm, regarding the total weight of the composition.

Accordingly, in a specific aspect of the present invention, the impurity may represent from 1 ppm to 100 ppm, preferably from 1 ppm to 50 ppm, and more preferably from 1 ppm to 10 ppm regarding the total weight of methyl dihydrojasmonate.

Advantageously, the methyl dihydrojasmonate of the present invention has a purity higher than 90%, preferably higher than 95%, more preferably higher than 96%, even more preferably higher than 99%, even more preferably higher than 99.5%, most preferably higher than 99.9%.

It is well-known by the person skilled in the art that the organoleptic properties of a flavoring substance may depend from the presence and the quantity of some impurities. That is why the manufacturing method is critical for the flavor of the final compound. Advantageously, it was discovered that the methyl dihydrojasmonate of the present invention displays satisfactory organoleptic properties. It is worth mentioning that the organoleptic profile of the methyl dihydrojasmonate of the present invention is equivalent to the organoleptic profile of methyl dihydrojasmonate from fossil origin in particular synthetically prepared from adipic acid, cyclopentene or dicylopentadiene.

### Cyclopentanone

The invention relates to cyclopentanone having a bio-based carbon content higher or equal to 50%, preferably higher or equal to 70%. Generally cyclopentanone according to the present invention has a bio-based carbon content lower or equal to 105%, preferably equal to lower to 100%, more preferably lower than 100%.

According to another aspect, the number of carbon atoms from bio-based origin of cyclopentanone according to the present invention is higher or equal to 2, preferably higher or equal to 3, more preferably higher or equal to 4. In a specific embodiment all carbon atoms of cyclopentanone according to the present invention are from bio-based origin.

The present invention further relates to a composition comprising, or consisting essentially of cyclopentanone, whose bio-based carbon content is higher or equal to 50%, preferably higher or equal to 70%.

The bio-based cyclopentanone of the present invention may preferably display a mean isotopic ¹³C deviation of from −25 to −8‰, preferably from −24‰ to −9‰, more preferably from −15‰ to −9‰.

Said cyclopentanone may represent the major compound of the composition according to the present invention. Accordingly said cyclopentanone may represent more than 50%, preferably more than 70%, more preferably more than 80% regarding the total weight of the composition. In a more preferred aspect of the present invention the said cyclopentanone may represent more than 90%, preferably more than 95%, more preferably more than 96%, more preferably more than 99%, most preferably more than 99.5% regarding the total weight of the composition. The impurity may represent from 1 ppm to 5000 ppm, preferably from 1 ppm to 500 ppm, more preferably from 1 ppm to 50 ppm, most preferably from 1 ppm to 20 ppm, regarding the total weight of the composition.

Accordingly in a specific aspect of the present invention, the impurity may represent from 1 ppm to 100 ppm, preferably from 1 ppm to 50 ppm, and more preferably from 1 ppm to 10 ppm regarding the total weight of cyclopentanone.

Advantageously, the cyclopentanone of the present invention has a purity higher than 90%, preferably higher than 95%, more preferably higher than 96%, even more preferably higher than 99%, even more preferably higher than 99.5%, most preferably higher than 99.9%.

According to a particular embodiment of the present invention, the cyclopentanone has a purity comprised between 90% and 99.5% and comprises at least one compound selected from cyclopentanol and tetrahydrofurfuryl alcohol.

The invention further relates to a composition comprising:
90% to 99.5% by weight, preferably from 92% to 98%, more preferably from 94% to 96%, most preferably about 95% of cyclopentanone, and
   from 0.5 to 10% by weight, preferably from 2% to 8%, more preferably from 4% to 6%, most preferably about 5% of at least one compound selected from cyclopentanol and tetrahydrofurfuryl alcohol. Preferably, the composition of the present invention comprises cyclopentanone wherein the cyclopentanone has a bio-based carbon content higher or equal to 50% or wherein the number of carbon atoms from bio-based origin is higher or equal to 2.

Preferably, the composition of the present invention comprises cyclopentanone wherein the cyclopentanone has a bio-based carbon content higher or equal to 70%, and/or wherein the number of carbon atoms from bio-based origin is higher or equal to 3, preferably higher or equal to 4. Preferably, the composition of the present invention comprises cyclopentanone wherein the cyclopentanone displays a mean isotopic ¹³C deviation of from −25 to −8‰, more preferably from −24 to −9‰, more preferably from −15‰ to −9‰.

It is well-known by the person skilled in the art that the organoleptic properties of a flavoring substance may depend from the presence and the quantity of some impurities. That is why the manufacturing method is critical for the flavor of the final compound. Advantageously, it was discovered that the cyclopentanone of the present invention displays satisfactory properties in particular for its use in the preparation flavor and/or fragrances, in particular methyl dihydrojasmonate and for its use in electronics, in particular as a solvent for the manufacturing of semiconductors.

### Manufacturing Process

In another aspect, the present invention relates to a process for the preparation of a methyl dihydrojasmonate and/or a cyclopentanone and/or a composition according to the present invention from furfuryl alcohol whose bio-based carbon content is higher or equal to 50%. The furfuryl alcohol used for the preparation of the methyl dihydrojasmonate and/or a cyclopentanone according to the present invention is a bio-based furfuryl alcohol.

Furfuryl alcohol having a bio-based carbon content above 50% is hereafter also called “bio-based furfuryl alcohol”. Bio-based furfuryl alcohol according to the invention may have a bio-based carbon content above 70%, preferably above 85%, more preferably above 90%, more preferably above 95%, more preferably above 98%, and more preferably above 99%. Bio-based furfuryl alcohol is a commercial product. Generally the bio-based furfuryl alcohol according to the invention may have a bio-based carbon content lower or equal to 105%, preferably lower or equal to 103%, more preferably lower or equal to 100%. Because of the natural sourcing, the raw furfuryl alcohol may contain some impurities. Said impurities may be specific to the origin of the compound. Typically, the content of each impurity in the bio-based furfuryl alcohol may be comprised between 0.005 and 0.1%, more preferably between 0.01 and 0.08%.

It could be naturally obtained from naturally occurring substrates like furfural, by different methods such as hydrogenation. For instance, furfural may be obtained from wood hemicellulose by degradation into xylose and then dehydration of xylose into furfural. Current commercial processes are usually starting from wood or biomass wastes like corn stovers. Because of the natural sourcing, the raw furfural may contain some impurities. Said impurities may be specific to the origin of the compound. Typically, the content of each impurity in the bio-based furfural may be comprised between 0.005 and 0.1%, more preferably between 0.01 and 0.08%.

The bio-based furfuryl alcohol used in the present invention may preferably display a mean isotopic ¹³C deviation of from −25 to −12‰, more preferably from −24 to −14‰.

According to another particular embodiment, the bio-based furfuryl alcohol used in the present invention may display a mean isotopic deviation of from −15‰ to −8‰, preferably from −13‰ to −9‰.

### Pentanal

Pentanal may be bio-based pentanal or non-bio-based pentanal.

According to a preferred embodiment of the present invention, pentanal has a bio-based carbon content above 50% is hereafter also called “bio-based pentanal”. Bio-based pentanal according to the invention may have a bio-based carbon content above 60%, preferably between 75% and 105%, more preferably between 90% and 103%, more preferably between 95% and 100%, more preferably between 98% and 100%, and more preferably between 99% and 100%.

The pentanal may be from fossil origin. According to a particular embodiment, the pentanal may be upcycled, for example the pentanal may be obtained from wastes or by-products of plastic production.

Because of the natural sourcing, the raw pentanal may contain some impurities. Said impurities may be specific to the origin of the compound. Typically, the content of each impurity in the bio-based pentanal may be comprised between 0.005 and 0.1%, more preferably between 0.01 and 0.08%.

### Dimethyl Malonate

Dimethyl malonate may be bio-based dimethyl malonate or non-bio-based dimethyl malonate. According to a preferred embodiment of the present invention, dimethyl malonate has a bio-based carbon content above 50% is hereafter also called “bio-based dimethyl malonate”. Bio-based dimethyl malonate according to the invention may have a bio-based carbon content above 60%, preferably between 75% and 105%, more preferably between 90% and 103%, more preferably between 95% and 100%, more preferably between 98% and 100%, and more preferably between 99% and 100%.

Dimethyl malonate may be obtained from malonic acid, preferably malonic acid displays a mean isotopic ¹³C deviation of from −22 to −17‰.

The bio-based dimethyl malonate may be obtained by fermentation of glucose to produce malonic acid, see for example WO 2021/042058, followed by esterification with methanol, preferably bio-based methanol.

### Step (a)—Hydrogenation

According to the present invention, the process for the preparation of cyclopentanone or a composition of the present invention comprises:
a step (a) wherein bio-based furfuryl alcohol is hydrogenated.

According to the present invention, the process for the preparation of methyl dihydrojasmonate comprises:
a step (a) wherein bio-based furfuryl alcohol is hydrogenated.

After step (a) a bio-based cyclopentanone or composition comprising bio-based cyclopentanone is obtained.

Step (a) is a hydrogenation step. Step (a) may be conducted according to the conditions described in Appl. Catal. B: Environ. 2014. 154-155, 294.

Step (a) may give rise to some impurities, namely cyclopentanol and/or tetrahydrofurfuryl alcohol.

Step (a) is generally conducted in aqueous solvent, preferably step (a) is conducted in water.

The aqueous solvent may be a mixture of water and a water soluble organic solvent, preferably selected from alcohol, ethyl acetate, more preferably selected from the group consisting of ethanol, isopropanol, and ethyl acetate.

Generally the concentration of furfuryl alcohol in the aqueous solvent is below 30% by weight, preferably below 20%, more preferably below 10% and still more preferably below 8% by weight.

Generally, step (a) is conducted at a pH comprised between 2 and 6, preferably comprised between 3 and 5.

Generally step (a) is conducted under an H₂ atmosphere, preferably at a pressure comprised between 5 and 50 bar, more preferably comprised between 10 and 40 bar, still more preferably comprised between 20 and 30 bar.

Generally step (a) is conducted at a temperature comprised between 120° C. and 200° C., preferably at a temperature comprised between 150° C. and 180° C.

The hydrogenation reaction (step (a)) is generally catalysed, preferably the catalyst is a metal catalyst, wherein the metal is preferably selected from Cu, Pd, Pt, Ni, Au, Rh, Ru. The catalyst may preferably be a nickel based catalyst, preferably Raney-nickel.

Generally the amount of catalyst is lower or equal to 20 wt %, preferably lower or equal to 10 wt %, still more preferably lower or equal to 1 wt %. Generally the amount of catalyst is higher or equal to 0.001 wt %, preferably lower or equal to 0.01 wt %, still more preferably lower or equal to 0.1 wt %.

Advantageously, firstly, the furfuryl alcohol is hydrogenated to form a compound of formula (I) which is then hydrolysed to form a compound of formula (II). The compound of formula (II) is then under the same reaction condition hydrogenated to form cyclopentanone. This one-pot process is shown in the scheme below:

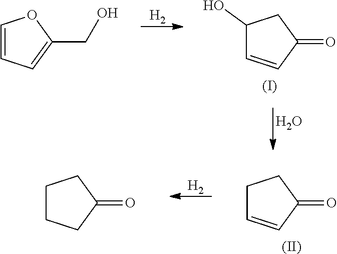

After the hydrogenation reaction, the bio-based cyclopentanone obtained may be separated from the reaction mixture via standard separation techniques.

After step (a), an aqueous composition comprising:
from 1% to 10% by weight, preferably from 1.5% to 5%, more preferably from 2% to 4% of cyclopentanone,
from 0.01% to 1% by weight, preferably from 0.05% to 0.5% of cyclopentanol, and
from 0.001% to 0.5% by weight, preferably from 0.01% to 0.1% of tetrahydrofurfuryl alcohol.

The composition obtained after step (a) may be purified by azeotropic distillation of a composition comprising water, cyclopentanone and cyclopentanol. The distilled azeotrope is then further purified by decantation. The organic phase is then further distiller in order to obtain a purified cyclopentanone.

The distillation may be performed at atmospheric pressure or under reduced pressure, preferably the distillation is performed under reduced pressure. The use of reduce pressure prevents degradation of the cyclopentanone.

Alternatively, the reaction mixture obtained after the hydrogenation step (a) may be used in its existing form, for example in step (b).

### Step (b)—Aldol Reaction

According to the present invention, the process for the preparation of methyl dihydrojasmonate further comprises:
a step (b) wherein bio-based cyclopentanone is reacted with pentanal.

Preferably step (b) is conducted after step (a). The pentanal may be a bio-based pentanal or a pentanal of fossil origin, for example upcycled. According to a particular embodiment, the pentanal may be obtained from a fatty acid, for example mystiric acid or lauric acid.

Step (b) is an aldol condensation. Step (b) may be conducted according to the conditions described in U.S. Pat. No. 4,260,830 or U.S. Pat. No. 3,158,644.

Step (b) is generally conducted in the presence of a base, preferably NaOH. The amount of pentanal is generally comprised between 0.5 and 1.2 equivalents. Step (b) is generally conducted in water.

Step (b) may be conducted at ambient temperature.

After dehydration and isomerisation of the carbon-carbon double, a compound of formula (III) is obtained.

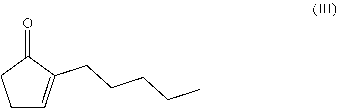

According to the present invention, the compound of formula (III) may have a bio-based carbon content higher or equal to 50%, preferably higher or equal to 70%. Generally the compound of formula (III) according to the present invention has a bio-based carbon content lower or equal to 105%, preferably equal to lower to 100%, more preferably lower than 100%.

According to another aspect, the number of carbon atoms from bio-based origin of the compound of formula (III) according to the present invention is higher or equal to 2, preferably higher or equal to 3, more preferably higher or equal to 4, preferably higher or equal to 5. In a specific embodiment all carbon atoms of the compound of formula (III) according to the present invention are from bio-based origin.

### Step (c)—Michael Addition

According to the present invention, the process for the preparation of methyl dihydrojasmonate further comprises:
a step (c) wherein the compound of formula (III) is reacted with dimethyl malonate.

Preferably step (c) is conducted after step (b), preferably conducted after step (a) and (b). The dimethyl malonate may be a bio-based dimethyl malonate or a dimethyl malonate of fossil origin. The bio-based dimethyl malonate may be obtained by fermentation of glucose, see for example WO 2021/042058, followed by esterification with bio-based methanol.

Step (c) is a Michael addition. Step (c) may be conducted according to the conditions described in U.S. Pat. No. 4,260,830.

Step (c) is generally conducted in the presence of a base, preferably NaOH. The amount of dimethyl malonate is generally comprised between 1.0 and 1.5 equivalents. Step (c) is generally conducted in a solvent comprising an alcohol, preferably methanol.

Step (c) may be conducted at a temperature comprised between −10° C. and ambient temperature. After decarboxylation, methyl dihydrojasmonate is obtained.

Generally a mixture of diastereoisomers is obtained (cis and trans). Each diastereoisomer is obtained in a racemic mixture.

According to the present invention, the methyl dihydrojasmonate obtained may have a bio-based carbon content higher or equal to 30%, preferably higher or equal to 35%, more preferably higher or equal to 38%. Generally the methyl dihydrojasmonate according to the present invention has a bio-based carbon content lower or equal to 105%, preferably equal to lower to 100%, more preferably lower than 100%.

According to another aspect, the number of carbon atoms from bio-based origin of the methyl dihydrojasmonate according to the present invention is higher or equal to 2, preferably higher or equal to 3, more preferably higher or equal to 4, preferably higher or equal to 5. In a specific embodiment all carbon atoms of methyl dihydrojasmonate according to the present invention are from bio-based origin.

In another aspect of the present invention, the methyl dihydrojasmonate and/or cyclopentanone and/or composition obtainable by the process as disclosed above is a subject-matter of the present invention. These compounds differ from the compounds already known in the art by the fact that they are partly or wholly prepared from raw materials originating from natural or renewable sources.

This specificity of the methyl dihydrojasmonate and/or cyclopentanone and/or composition can be determined by a bio-based carbon content measure and/or a mean isotopic ¹³C deviation.

The methyl dihydrojasmonate and/or cyclopentanone and/or composition of the present invention may advantageously be used as a flavour or a fragrance or as a solvent for the manufacture of semiconductors. Preferably the methyl dihydrojasmonate and/or cyclopentanone and/or composition of the present invention may be used in industry, for instance in the food, pharmaceutical or cosmetics industry, in particular for example for manufacturing fragrances.

Another object of the present invention relates to a composition comprising a methyl dihydrojasmonate and/or cyclopentanone and/or composition of the present invention preferably selected from the group consisting of food products, beverages, cosmetic formulations, pharmaceutical formulations and fragrances. The disclosure of all patent applications, and publications cited herein are hereby incorporated by reference, to the extent that they provide exemplary, procedural or other details supplementary to those set forth herein. Should the disclosure of any of the patents, patent applications, and publications that are incorporated herein by reference conflict with the present specification to the extent that it might render a term unclear, the present specification shall take precedence.

Each and every claim is incorporated into the specification as an embodiment of the present invention. Thus, the claims are a further description and are an addition to the preferred embodiments of the present invention.

While preferred embodiments of this invention have been shown and described, modifications thereof can be made by one skilled in the art without departing from the spirit or teaching of this invention. The embodiments described herein are exemplary only and are not limiting. Many variations and modifications of systems and methods are possible and are within the scope of the invention.

Accordingly, the scope of protection is not limited by the description set out above, but is only limited by the claims which follow, that scope including all equivalents of the subject matter of the claims.

Should the disclosure of any patents, patent applications, and publications which are incorporated herein by reference conflict with the description of the present application to the extent that it may render a term unclear, the present description shall take precedence.

### EXAMPLES

### Example 1: Preparation of Bio-Based Cyclopentanone

3 g Bio-based furfuryl alcohol (100% of bio-based carbon content) was hydrogenated in 60 mL water in the presence of a nickel catalyst (30 mg). The hydrogen pressure is of 30 bar and the temperature of 160° C.

After 3-4 hours of stirring the reaction mixture is analyzed. Bio-based cyclopentanone is obtained and displays 100% of bio-based carbon content. Conversion of 100%.

### Example 2: Preparation of Bio-Based Methyl Dihydrojasmonate

Bio-based cyclopentanone (100% bio-based carbon content) of example 1 is reacted with pentanal to produce a compound of formula (III).

The compound of formula (III) is then reacted with 100% bio-based dimethyl malonate having a bio-based carbon content of 100%.

Methyl dihydrojasmonate is obtained and displays a bio-based carbon content of 62%.

1. Methyl dihydrojasmonate having:
a bio-based carbon content greater than or equal to 30%, and/or
a number of carbon atoms from bio-based origin greater than or equal to 2.

2. The methyl dihydrojasmonate according to claim 1 having;
a bio-based carbon content greater than or equal to 35%, and/or
a number of carbon atoms from bio-based origin greater than or equal to 5.

3. Cyclopentanone having:
a bio-based carbon content higher greater or equal to 50%, or
wherein the a number of carbon atoms from bio-based origin is higher greater than or equal to 2.

4. The cyclopentanone according to claim 3 having:
a bio-based carbon content higher or equal to 70%, and/or
wherein the a number of carbon atoms from bio-based origin is higher greater than or equal to 3, preferably higher or equal to 4.

5. The cyclopentanone according to claim 3 displaying a mean isotopic ¹³C deviation of from −25 to −8‰.

6. A composition comprising:
90% to 99.5% by weight, preferably from 92% to 98%, more preferably from 94% to 96%, most preferably about 95% of cyclopentanone, and
from 0.5 to 10% by weight, preferably from 2% to 8%, more preferably from 4% to 6%, most preferably about 5% of at least one compound selected from cyclopentanol and tetrahydrofurfuryl alcohol.

7. The composition according to claim 6, wherein the cyclopentanone has a bio-based carbon content higher or equal to 50%, or wherein the number of carbon atoms from bio-based origin is greater than or equal to 2.

8. The composition according to claim 7, wherein the cyclopentanone has a bio-based carbon content greater than or equal to 70%, and/or wherein the number of carbon atoms from bio-based origin is greater than or equal to 3.

9. The composition according to claim 6, wherein the cyclopentanone displays a mean isotopic ¹³C deviation of from −25 to −8‰.

10. A process for the preparation of methyl dihydrojasmonate, cyclopentanone, or the composition as defined in claim 6, the process comprising:
preparing the methyl dihydrojasmonate, the cyclopentanone, or the composition as defined in claim 6 from bio-based furfuryl alcohol comprising a bio-based carbon content is greater than or equal to 50%.

11. The process according to claim 10, further comprising a step (a) wherein bio-based furfuryl alcohol is hydrogenated.

12. The process according to claim 10, further comprising a step (b) wherein bio-based cyclopentanone is reacted with pentanal to form a compound of formula (III).

13. The process according to claim 12, further comprising a step (c) wherein the compound of formula (III) is reacted with dimethyl malonate.

14. A process for preparing an electronic, perfurmery, cosmetic, flavor, fragrance, and/or pharmaceutical composition, the process comprising:
preparing a flavor, fragrance, and/or an electronic composition comprising:
the composition as defined in claim 6; or
cyclopentanone having a bio-based carbon content or equal to 50% or a number of carbon atoms from bio-based origin greater than or equal to 2; or
preparing a flavor or fragrance composition comprising:
methyl dihydrojasmonate having a bio-based carbon content greater than or equal to 30% and/or a number of carbon atoms from bio-based origin greater than or equal to 2.

15. A composition comprising:
a methyl dihydrojasmonate having:
a bio-based carbon content higher or equal to 30%, and/or
a number of carbon atoms from bio-based origin greater than or equal to 2; and/or
cyclopentanone having:
a bio-based carbon content higher or equal to 50%, or
a number of carbon atoms from bio-based origin greater than or equal to 2; and/or
the composition as defined in claim 6.

16. The methyl dihydrojasmonate according to claim 1 having:
a bio-based carbon content greater than equal to 38%, and/or
a number of carbon atoms from bio-based origin greater than or equal to 7.

17. The cyclopentanone according to claim 3 having:
a bio-based carbon content higher or equal to 70%, and/or
a number of carbon atoms from bio-based origin greater than equal to 4.

18. The cyclopentanone according to claim 3 displaying a mean isotopic ¹³C deviation of from −24 to −9‰.

19. The composition of claim 6 comprising:
92% to 98% by weight of cyclopentanone, and
from 2 to 8% by weight of at least one compound selected from cyclopentanol and tetrahydrofurfuryl alcohol.

20. The composition of claim 6 comprising:
94% to 96% by weight of cyclopentanone, and
from 4 to 6% by weight of at least one compound selected from cyclopentanol and tetrahydrofurfuryl alcohol.
